(19) United States
(12) Patent Application Publication
(10) Pub. No.: US 2025/0128045 A1
(43) Pub. Date: Apr. 24, 2025

(54) PERCUTANEOUS BLOOD PUMP WITH MOTOR CONNECTION

(71) Applicant: Boston Scientific Scimed, Inc., Maple Grove, MN (US)

(72) Inventors: Darrin Dale Beekman, Osseo, MN (US); Maren Landree, Medina, MN (US); Benjamin Breidall, Eden Prairie, MN (US); Matthew Nelson Frost, New Brighton, MN (US)

(73) Assignee: Boston Scientific Scimed, Inc., Maple Grove, MN (US)

(21) Appl. No.: 18/923,829

(22) Filed: Oct. 23, 2024

Related U.S. Application Data
(60) Provisional application No. 63/592,262, filed on Oct. 23, 2023.

(51) Int. Cl. A61M 60/178; A61M 60/216

(52) U.S. Cl. CPC A61M 60/178; A61M 60/216; A61M 2210/125

(57) ABSTRACT

A percutaneous circulatory support device including a percutaneous blood pump coupled to a distal end region of an elongate shaft at a junction. The percutaneous blood pump is configured to pump blood through a housing of the percutaneous blood pump during use. The junction includes an end cap at a proximal end of the percutaneous blood pump surrounding and secured to a distal end region of the elongate shaft at the junction. The junction may include a fillet of material surrounding a proximal end region of the end cap and extending proximally thereof and surrounding an exterior surface of the elongate shaft. The proximal end region of the end cap may include an annular side wall with one or more apertures and/or one or more grooves extending into the annular side wall in which the fillet of material extends into.

### CROSS-REFERENCE TO RELATED APPLICATIONS

The present application claims the benefit of U.S. Provisional Patent Application Ser. No. 63/592,262, filed Oct. 23, 2023, the disclosure of which is incorporated herein by reference.

### TECHNICAL FIELD

The present disclosure pertains to a catheter including a percutaneous blood pump. More particularly, the present disclosure pertains a percutaneous circulatory support device including a connection between an elongate shaft of a catheter and a percutaneous blood pump secured thereto.

### BACKGROUND

Percutaneous mechanical circulatory support devices, such as blood pumps can provide transient support for hours or months of use in patients whose heart function or cardiac output is compromised. The percutaneous mechanical circulatory support devices may be sufficiently flexible to be navigated through the vasculature to a patient's heart. Such devices may be navigated through the aortic arch and placed across the aortic valve, for example. Various configurations of percutaneous mechanical circulatory support devices are known. However, there is an ongoing need to provide improved construction of percutaneous mechanical circulatory support devices.

### BRIEF SUMMARY

This disclosure provides design, material, manufacturing method, and use alternatives for medical devices, including percutaneous circulatory support devices and associated percutaneous blood pumps.

A first example is a percutaneous circulatory support device including a flexible elongate shaft and a percutaneous blood pump coupled to a distal end region of the elongate shaft at a junction. The percutaneous blood pump is configured to pump blood through a housing of the percutaneous blood pump during use. The junction includes an end cap at a proximal end of the percutaneous blood pump surrounding and secured to a distal end region of the elongate shaft at the junction.

Alternatively or additionally to any of the examples above, in another example, the end cap includes an annular side wall and one or more apertures extending into the annular side wall.

Alternatively or additionally to any of the examples above, in another example, the one or more apertures are through holes extending through an entire thickness of the side wall, the through holes having material of the end cap completely surrounding a periphery of the through holes.

Alternatively or additionally to any of the examples above, in another example, the one or more apertures are blind holes extending through only a portion of a thickness of the side wall.

Alternatively or additionally to any of the examples above, in another example, the one or more apertures are slots extending through an entire thickness of the side wall and opening out to a proximal end of the end cap.

Alternatively or additionally to any of the examples above, in another example, the percutaneous blood pump includes a blood inlet, a blood outlet, an impeller assembly positioned between the blood inlet and the blood outlet, and a motor housing positioned proximal of the impeller, the motor housing including a motor to rotationally drive an impeller of the impeller assembly to pump blood from the blood inlet to the blood outlet during use.

Alternatively or additionally to any of the examples above, in another example, the motor housing includes a main body surrounding the motor, and the end cap is a separate component fixed to the main body and extending proximally therefrom.

Alternatively or additionally to any of the examples above, in another example, the motor housing includes a main body surrounding the motor, and the end cap is formed monolithically with the main body.

Alternatively or additionally to any of the examples above, in another example, the end cap includes a distal end region having a first outer diameter and a proximal end region having a second outer diameter, the second outer diameter being less than the first outer diameter.

Alternatively or additionally to any of the examples above, in another example, the proximal end region of the end cap includes an annular side wall and one or more apertures extending into the annular side wall.

Alternatively or additionally to any of the examples above, in another example, the junction further includes a fillet of material surrounding the proximal end region of the end cap and extending proximally thereof and surrounding an exterior surface of the elongate shaft.

Alternatively or additionally to any of the examples above, in another example, the fillet of material extends into the one or more apertures.

Alternatively or additionally to any of the examples above, in another example, the end cap includes a shoulder between the proximal end region of the end cap and the distal end region of the end cap, wherein the fillet of material extends distal of the shoulder and surrounds a portion of the distal end region of the end cap.

Alternatively or additionally to any of the examples above, in another example, an outer circumferential surface of the proximal end region of the end cap includes one or more grooves, wherein the fillet of material extends into the one or more grooves.

Another example is a percutaneous circulatory support device including a flexible elongate shaft and a percutaneous blood pump coupled to a distal end region of the elongate shaft at a junction. The percutaneous blood pump includes a blood inlet, a blood outlet, an impeller assembly positioned between the blood inlet and the blood outlet, and a motor housing positioned proximal of the impeller. The motor housing includes a motor to rotationally drive an impeller of the impeller assembly to pump blood from the blood inlet to the blood outlet during use. The motor housing includes an end cap surrounding and secured to a distal end region of the elongate shaft at the junction.

Alternatively or additionally to any of the examples above, in another example, the end cap incudes an annular side wall and one or more apertures extending into the annular side wall.

Alternatively or additionally to any of the examples above, in another example, the one or more apertures are through holes extending through an entire thickness of the side wall, the through holes having material of the end cap completely surrounding a periphery of the through holes.

Alternatively or additionally to any of the examples above, in another example, the one or more apertures are blind holes extending through only a portion of a thickness of the side wall.

Alternatively or additionally to any of the examples above, in another example, the one or more apertures are slots extending through an entire thickness of the side wall and opening out to a proximal end of the end cap.

Alternatively or additionally to any of the examples above, in another example, the motor housing includes a main body surrounding the motor, and the end cap is a separate component fixed to the main body.

Alternatively or additionally to any of the examples above, in another example, the motor housing includes a main body surrounding the motor, and the end cap is formed monolithically with the main body.

Alternatively or additionally to any of the examples above, in another example, the end cap includes a distal end region having a first outer diameter and a proximal end region having a second outer diameter, the second outer diameter being less than the first outer diameter.

Alternatively or additionally to any of the examples above, in another example, the proximal end region of the end cap includes an annular side wall and one or more apertures extending into the annular side wall.

Alternatively or additionally to any of the examples above, in another example, the junction further includes a fillet of material surrounding the proximal end region of the end cap and extending proximally thereof and surrounding an exterior surface of the elongate shaft.

Alternatively or additionally to any of the examples above, in another example, the fillet of material extends into the one or more apertures.

Alternatively or additionally to any of the examples above, in another example, the end cap includes a shoulder between the proximal end region of the end cap and the distal end region of the end cap, wherein the fillet of material extends distal of the shoulder and surrounds a portion of the distal end region of the end cap.

Alternatively or additionally to any of the examples above, in another example, an outer circumferential surface of the proximal end region of the end cap includes one or more grooves, wherein the fillet of material extends into the one or more grooves.

Alternatively or additionally to any of the examples above, in another example, the one or more grooves extend circumferentially or helically around the outer circumferential surface.

Another example is a percutaneous circulatory support device including a flexible elongate shaft and a percutaneous blood pump coupled to a distal end region of the elongate shaft at a junction. The percutaneous blood pump is configured to pump blood through a housing of the percutaneous blood pump during use. The junction includes an end cap at a proximal end of the percutaneous blood pump surrounding and secured to a distal end region of the elongate shaft at the junction.

Alternatively or additionally to any of the examples above, in another example, the junction further includes a fillet of material surrounding a proximal end region of the end cap and extending proximally thereof and surrounding an exterior surface of the elongate shaft.

Alternatively or additionally to any of the examples above, in another example, the proximal end region of the end cap includes an annular side wall and one or more apertures extending into the annular side wall.

Alternatively or additionally to any of the examples above, in another example, the fillet of material extends into the one or more apertures and is bonded to the elongate shaft.

Alternatively or additionally to any of the examples above, in another example, an outer circumferential surface of the proximal end region of the end cap includes one or more grooves, wherein the fillet of material extends into the one or more grooves.

Another example is a method of manufacturing a percutaneous circulatory support device. The method includes inserting a distal end region of an elongate shaft of the percutaneous circulatory support device into a proximal end region of an end cap of a percutaneous blood pump of the percutaneous circulatory support device, and applying a fillet of material over the proximal end region of the end cap and over a portion of the elongate shaft extending proximally from the end cap to form a junction between the end cap and the elongate shaft.

Alternatively or additionally to any of the examples above, in another example, the end cap incudes an annular side wall and one or more apertures extending into the annular side wall.

Alternatively or additionally to any of the examples above, in another example, the one or more apertures are through holes extending through an entire thickness of the side wall, the through holes having material of the end cap completely surrounding a periphery of the through holes.

Alternatively or additionally to any of the examples above, in another example, the one or more apertures are blind holes extending through only a portion of a thickness of the side wall.

Alternatively or additionally to any of the examples above, in another example, the one or more apertures are slots extending through an entire thickness of the side wall and opening out to a proximal end of the end cap.

Alternatively or additionally to any of the examples above, in another example, the fillet of material extends into the one or more apertures and is bonded to the elongate shaft.

Alternatively or additionally to any of the examples above, in another example, an outer circumferential surface of the proximal end region of the end cap includes one or more grooves, wherein the fillet of material extends into the one or more grooves.

The above summary of some embodiments is not intended to describe each disclosed embodiment or every implementation of the present disclosure. The Figures, and Detailed Description, which follow, more particularly exemplify some of these embodiments.

### BRIEF DESCRIPTION OF THE DRAWINGS

The disclosure may be more completely understood in consideration of the following detailed description in connection with the accompanying drawings, in which:

FIG. 1 is a perspective view of an exemplary percutaneous circulatory support device including a percutaneous blood pump;

FIG. 2 shows the distal end region of the percutaneous circulatory support device of FIG. 1 including the percutaneous blood pump;

FIG. 3 is a side view of a portion of the percutaneous blood pump of FIG. 1;

FIG. 4 is a cross-sectional view of the portion of the percutaneous blood pump of FIG. 3;

FIG. 5 is an enlarged view of a connection between the percutaneous blood pump and the elongate shaft of the percutaneous circulatory support device of FIG. 1;

FIG. 6 is a perspective view of an end cap of the connection shown in FIG. 5;

FIG. 7 is a cross-sectional view of the end cap of FIG. 6;

FIG. 8 is a longitudinal cross-sectional view of the connection between the end cap and the elongate shaft of the percutaneous circulatory support device;

FIGS. 8A-8C depict one method of forming the connection between the end cap and the elongate shaft of the percutaneous circulatory support device; and

FIG. 9 is an alternative configuration of an end cap for a connection to the elongate shaft of the percutaneous circulatory support device.

While the disclosure is amenable to various modifications and alternative forms, specifics thereof have been shown by way of example in the drawings and will be described in detail. It should be understood, however, that the intention is not to limit the disclosure to the particular embodiments described. On the contrary, the intention is to cover all modifications, equivalents, and alternatives falling within the spirit and scope of the disclosure.

### DETAILED DESCRIPTION

For the following defined terms, these definitions shall be applied, unless a different definition is given in the claims or elsewhere in this specification.

All numeric values are herein assumed to be modified by the term “about,” whether or not explicitly indicated. The term “about” generally refers to a range of numbers that one of skill in the art would consider equivalent to the recited value (i.e., having the same function or result). In many instances, the terms “about” may include numbers that are rounded to the nearest significant figure.

The recitation of numerical ranges by endpoints includes all numbers within that range (e.g. 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

It is noted that references in the specification to “an embodiment”, “some embodiments”, “other embodiments”, etc., indicate that the embodiment described may include one or more particular features, structures, and/or characteristics. However, such recitations do not necessarily mean that all embodiments include the particular features, structures, and/or characteristics. Additionally, when particular features, structures, and/or characteristics are described in connection with one embodiment, it should be understood that such features, structures, and/or characteristics may also be used connection with other embodiments whether or not explicitly described unless clearly stated to the contrary.

The following detailed description should be read with reference to the drawings in which similar structures in different drawings are numbered the same. The drawings, which are not necessarily to scale, depict illustrative embodiments and are not intended to limit the scope of the disclosure.

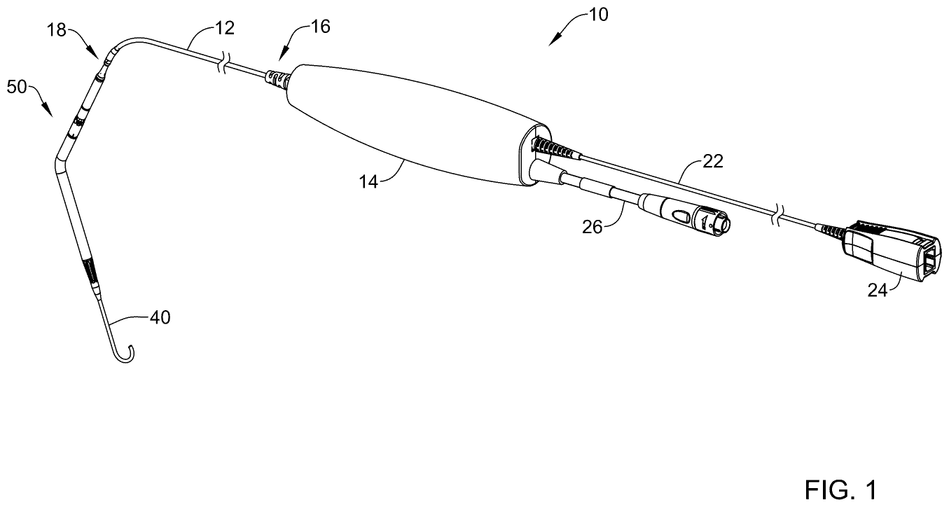

FIG. 1 illustrates a perspective view of a percutaneous circulatory support device 10 including a percutaneous blood pump 50 located at a distal end region thereof. The percutaneous circulatory support device 10 may be coupled to or include the blood pump 50, with an elongate shaft 12 of the percutaneous circulatory support device 10 extending proximally from the percutaneous blood pump 50 and a distal tip 40 extending distally from the blood pump 50. For instance, a proximal end 16 of the elongate shaft 12 may be coupled to a junction housing 14 and a distal end 18 of the elongate shaft 12 may be coupled to the percutaneous blood pump 50. An electrical cable 22 may extend from the junction housing 14 to a connector 24 at a proximal end thereof. The connector 24 may be configured to be connected to a controller (not shown) for controlling the blood pump 50, such as providing electrical power to the blood pump 50. The percutaneous circulatory support device 10 may also include an extension 26 connectable to the controller for sending and/or receiving signals, such as from one or more sensors during operation of the blood pump 50.

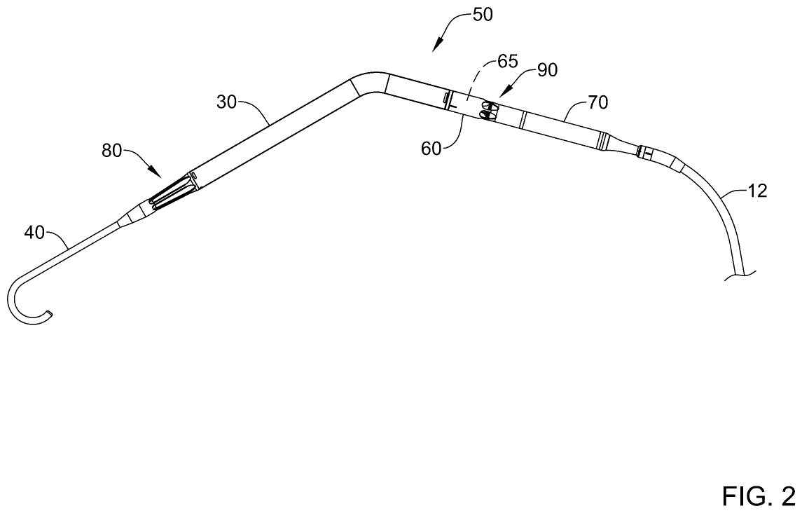

Additional features of the blood pump 50 are illustrated in FIG. 2. The blood pump 50 may generally include a flexible cannula 30, an impeller housing 60, and a motor housing 70. In some embodiments, the flexible cannula 30, the impeller housing 60 and/or the motor housing 70 may be integrally or monolithically constructed. In other instances, the flexible cannula 30, the impeller housing 60 and/or the motor housing 70 may be separate components. The impeller housing 60 carries an impeller assembly 65 therein. The impeller assembly 65 may include an impeller, secured to an impeller shaft, that rotate relative to the impeller housing 60 to drive blood through the blood pump 50. In some embodiments, the impeller shaft and the impeller of the impeller assembly 65 may be integrally formed, whereas, in other embodiments the impeller shaft and the impeller may be separate components.

Rotation of the impeller causes blood to flow from a blood inlet 80 of the blood pump 50, such as at a distal end of the flexible cannula 30, through the flexible cannula 30 and the impeller housing 60, and out of a blood outlet 90 proximal of the impeller, such as through a sidewall formed on the impeller housing 60. In some instances, the blood inlet 80 may include a plurality of blood inlet windows arranged around a circumference of the blood pump 50 (e.g., the flexible cannula 30). In some instances, the blood outlet 90 may include a plurality of blood outflow windows arranged around a circumference of the impeller housing 60. In other embodiments, the inlet 80 and/or the outlet 90 may be formed on other portions of the blood pump 50.

With continued reference to FIG. 2, the motor housing 70 carries a motor configured to rotatably drive the impeller of the impeller assembly 65 relative to the impeller housing 60. Electrical power may be supplied to the motor through wiring extending through the elongate shaft 12, for example. In some instances, the motor may be physically connected to the impeller. For example, in some embodiments the impeller may be mounted on the drive shaft of the motor. In other embodiments, the impeller shaft may be directly or indirectly coupled to the drive shaft of the motor. In some instances, the drive assembly may include a magnetic coupling between the motor and the impeller. For example, a driving magnet may be mounted on the drive shaft of the motor. Rotation of the driving magnet causes rotation of a driven magnet, which is connected to the impeller assembly 65. More specifically, in embodiments incorporating an impeller shaft, the impeller shaft and the impeller of the impeller assembly 65 are configured to rotate with the driven magnet. In other embodiments, the motor may be coupled to the impeller assembly 65 via other components.

The blood pump 50 may be guided over a guidewire during introduction of the blood pump 50 into the vasculature of a patient. For instance, a guidewire, inserted through a guidewire lumen of the distal tip 40, may be advanced proximally along the impeller assembly 65 and out through one of the outflow windows of the blood outlet 90 within the lumen of the guidewire loading aid 44. With the guidewire tracked through the blood pump 50, the percutaneous circulatory support device 10 may be advanced over the guidewire into a vasculature.

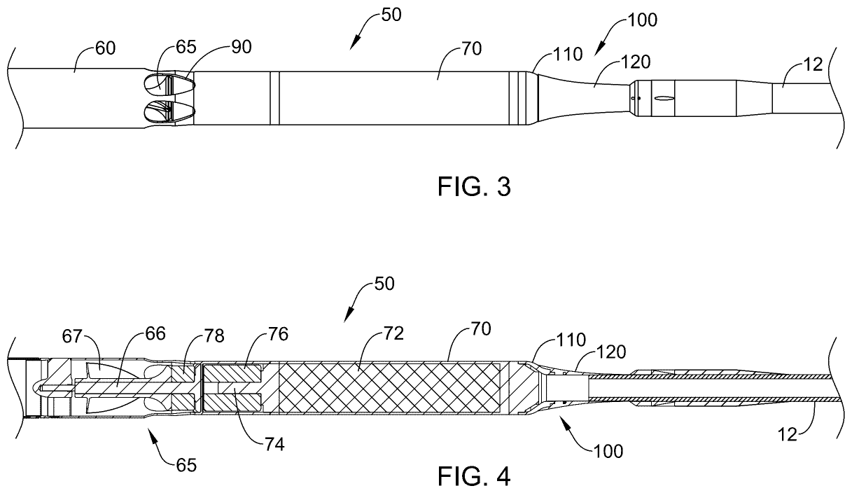

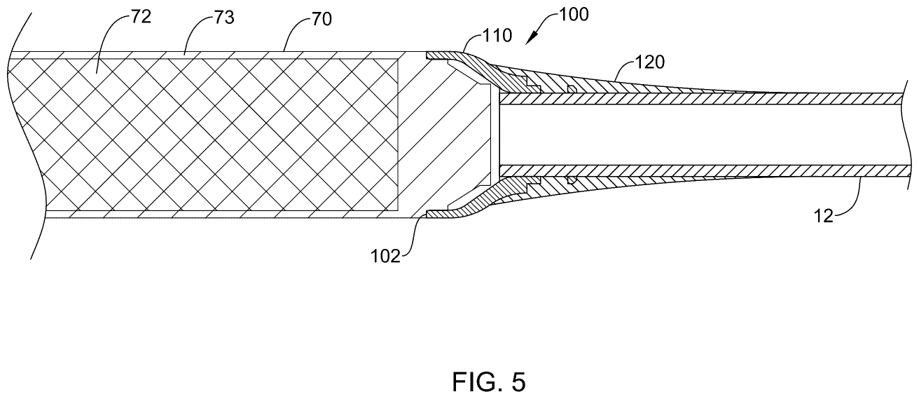

FIG. 3 is a side view of a portion of the percutaneous circulatory support device 10 illustrating a portion of the percutaneous blood pump 50 connected to the elongate shaft 12 at a junction 100. A proximal end of the impeller of the impeller assembly 65 is also visible through the outflow windows of the blood outlet 90. FIG. 4 is a cross-sectional view of the portion of the percutaneous circulatory support device 10 of FIG. 3, including the junction 100 between the percutaneous blood pump 50 and the elongate shaft 12 of the percutaneous circulatory support device 10. The junction 100 may be configured to mechanically connect the motor housing 70 (e.g., a metallic motor housing) of the blood pump 50, or other component of the blood pump 50, to the distal end region of the elongate shaft 12 (e.g., a polymeric tubular member).

The junction 100 may include an end cap 110 having a proximal end region surrounding the distal end region of the elongate shaft 12, and a fillet of material 120 surrounding the proximal end region of the end cap 110 and extending proximally therefrom. The fillet of material 120 may extend proximal of the end cap 110 and surround a portion of the elongate shaft 12 extending proximal of the end cap 110. Further details of the junction 100 are described herein.

As shown in FIG. 4, the impeller assembly 65 includes a drive shaft 66 and an impeller 67 coupled thereto, where the drive shaft 66 is configured to rotate with the impeller 67. As shown, the drive shaft 66 is at least partially disposed within the impeller 67. The impeller assembly 65 further includes a driven magnet 78 coupled to, and at least partially surrounding, the drive shaft 66 and/or the impeller 67. The driven magnet 78 may be any type of magnetic rotor capable of being driven by a driving magnet assembly. In this manner, as a magnetic field is applied to the driven magnet 78 by the driving magnet assembly, the driven magnet 78 rotates, causing the drive shaft 66 and impeller 67 to rotate.

As shown in FIG. 4, the driving magnet assembly includes a driving magnet 76 coupled to a drive line 74 configured to transfer torque from the motor 72 to the driving magnet 76. The motor housing 70 may be configured to hermetically seal the driving magnet 76 and the motor within the motor housing 70, and thus fluidly isolate the driving magnet 76 from the driven magnet 78. Electrical wires, not shown, may extend through a lumen of the elongate shaft 12 to provide electrical power to the motor 72. In other instances, the drive line 74 may be directly coupled to the impeller 67 and/or drive shaft 66, or other driven trains may be provided to transfer torque from the motor 72 to the impeller 67.

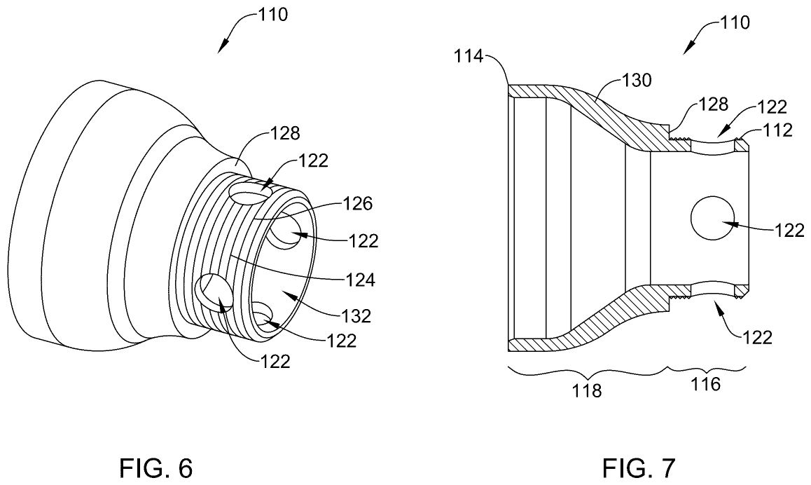

FIG. 5 is an enlarged view of a connection between the blood pump 50 and the elongate shaft 12 of the percutaneous circulatory support device 10. As shown in FIG. 5, the end cap 110 of the junction 100 may be formed as a separate component from the main body 73 of the motor housing 70, which surrounds the motor 72, and extend proximally therefrom. As such, the end cap 110 may be fixed to the main body 73 of the motor housing 70 in any desired fashion. For example, in some instances, each of the main body 73 and the end cap 110 may be formed of a metal material, welded together at a bond joint 102. In other instances, the main body 73 and the end cap 110 may be adhesive bonded, or otherwise fixed together at the bond joint 102, with the end cap 110 extending proximally therefrom.

The distal end region of the elongate shaft 12 may extend into the end cap 110 such that the end cap 110 overlaps with the distal end region of the elongate shaft 12. The fillet of material 120, which surrounds the proximal end region of the end cap 110, may taper proximally from a larger outer diameter at the distal end of the fillet of material 120 to a smaller outer diameter at the proximal end of the fillet of material 120.

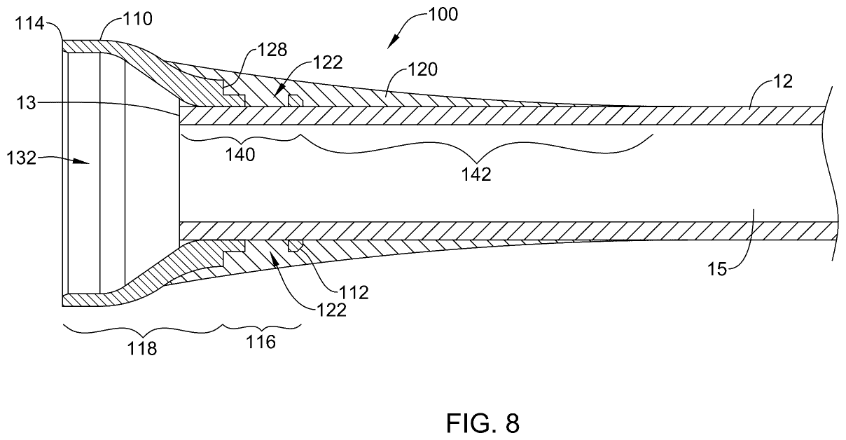

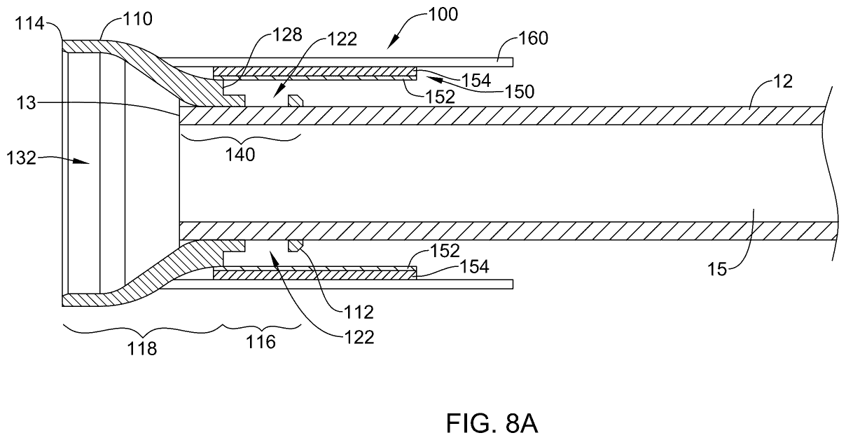

Additional details of the end cap 110 are shown in FIGS. 6 and 7. The end cap 110 may be a generally annular member having an annular side wall 130 defining a central lumen or bore 132 extending through the end cap 110 from a proximal end 112 to a distal end 114 of the end cap 110. The end cap 110 may including a proximal end region 116 extending to the proximal end 112 and a distal end region 118 extending to the distal end 114. The distal end region 118 may have a first outer diameter and the proximal end region 116 may have a second outer diameter. The second outer diameter (i.e., the outer diameter at the proximal end 112) may be less than the first outer diameter (i.e., the outer diameter at the distal end 114). In some instances, the end cap 110 may include a shoulder 128 between the proximal end region 116 and the distal end region 118.

The annular side wall 130 may include one or more, or a plurality of apertures 122 extending into the side wall 130 from an outer surface of the annular side wall 130. For example, the proximal end region 116 may include one or more, or a plurality of apertures 122. The apertures 122 may be symmetrically or asymmetrically arranged around a circumference of the end cap 110. In the illustrative embodiment the end cap 110 includes four apertures 122 symmetrically arranged around the circumference of the end cap 110 with each aperture 122 spaced about 90 degrees apart. In some instances, the one or more, a plurality of apertures 122 are through holes extending through the entire thickness of the side wall 130 from an outer surface of the side wall 130 to an inner surface of the side wall 130, with material of the end cap 110 completely surrounding a periphery of each of the through holes. In other instances, the one or more, or plurality of apertures 122 may be blind holes extending through only a portion of the thickness of the side wall 130. In yet other instances, the one or more, or plurality of apertures 122 may be slots extending through the entire thickness of the side wall and opening out to the proximal end 112 of the end cap 110.

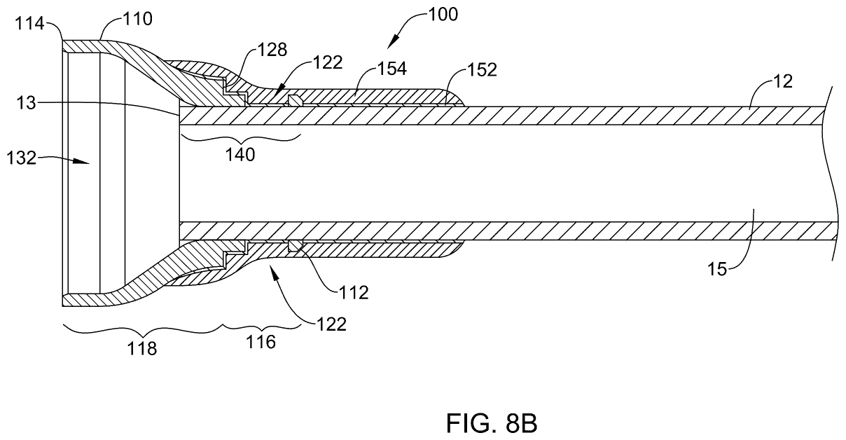

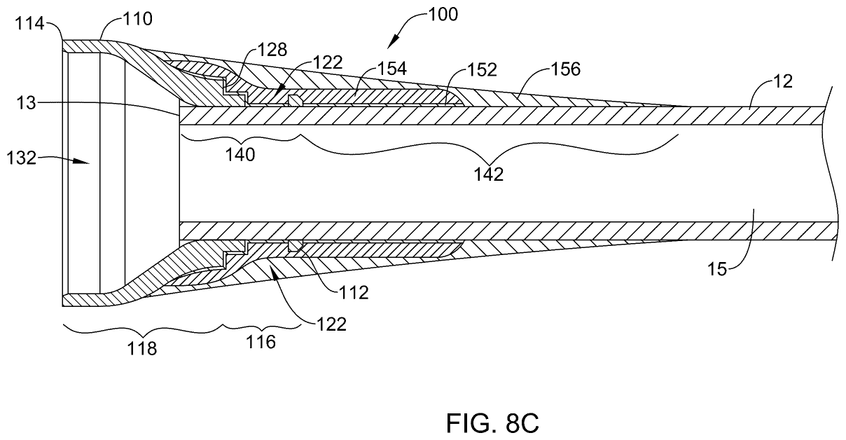

Furthermore, an outer circumferential surface 124 of the proximal end region 116 of the end cap 110 may include a roughened or textured surface to enhance the mechanical connection between the fillet of material 120 and the end cap 110. For example, the outer circumferential surface 124 may include one or more, or a plurality of grooves 126 formed therein, providing rigids and valleys along the outer circumferential surface 124. In some instances, the grooves 126 may extend circumferentially and/or helically around the outer circumferential surface 124. The grooves 126 may extend continuously or discontinuously around the outer circumferential surface 124. Turning to FIG. 8, the fillet of material 120 may surround the proximal end region 116 of the end cap 110 such that material of the fillet of material 120 extends into the grooves 126 to enhance the mechanical interlock between the fillet of material 120 and the end cap 110. In other instances, the roughened or textured surface of the outer circumferential surface 124 may include a plurality of dimples or recesses, notches, an etched surface, such as cross-hatched grooves, raised protuberances, or other surface characteristics formed in the outer circumferential surface 124 to provide the outer circumferential surface 124 with greater surface area for bonding to or otherwise engaging the fillet of material 120.

As shown in FIG. 8, a distal end region 140 of the elongate shaft 12 may extend into the lumen 132 of the end cap 110 such that the proximal end region 116 of the end cap 110 surrounds the distal end region 140 of the elongate shaft 12. The distal end region 118 of the end cap 110 may extend distally beyond the distal end 13 of the elongate shaft 12 with the distal end 114 of the end cap 110 located distal of the distal end 13 of the elongate shaft 12. The lumen 15 of the elongate shaft 12 may open into the lumen 132 of the end cap 110.

The fillet of material 120 may surround the proximal end region 116 of the end cap 110 and extend proximal of the end cap 110 and surround and be bonded to an outer surface of a portion 142 of the elongate shaft 12 extending proximally from the end cap 110. In some instances, the fillet of material 120 may extend distal of the shoulder 128 of the end cap 110 and also surround a portion of the distal end region 118 of the end cap 110.

The fillet of material 120 may extend into the apertures 122 of the end cap 110 such that material of the fillet of material 120 contacts the outer surface of the elongate shaft 12 through the apertures 122 and is bonded thereto. For example, the fillet of material 120 may be an adhesive material, such as a UV curable adhesive, applied over the end cap 110 and the elongate shaft 12 to secure the end cap 110 to the elongate shaft 12. In other instances, the fillet of material 120 may be a polymer material flowed over the end cap 110 and the elongate shaft 12 during a re-flow process to secure the end cap 110 to the elongate shaft 12. In some instances, the re-flow process may include applying multiple layers of polymer during multiple steps. The portion of the material of the fillet of material 120 flowing or extending into the apertures 122 may enhance the mechanical bond with the end cap 110, forming a mechanical interlock with the end cap 110.

During the manufacture of the junction 100 between the end cap 110 and the elongate shaft 12, the distal end region 140 of the elongate shaft 12 may be inserted into the proximal end region 116 of the end cap 110. The fillet of material 120 may then be applied over the proximal end region 116 of the end cap 110 and over the outer surface of the portion 142 of the elongate shaft 12 extending proximally from the end cap 110. The fillet of material 120 may flow into the apertures 122 and/or grooves 126 in the proximal end region 116 of the end cap 110, and thereafter solidify to interlock with the end cap 110. The outer diameter of the fillet of material 120 may taper proximally down to the outer diameter of the elongate shaft 12 to form a smooth transition from the outer diameter of the distal end region 118 of the end cap 110 to the outer diameter of the elongate shaft 12.

FIGS. 8A-8C illustrate one exemplary process of forming the fillet of material 120. As shown in FIG. 8A, a polymer tube 150, such as a coextruded tube, may be disposed over the proximal end region 116 of the end cap 110 with the tube 150 surrounding the proximal end region 116 of the end cap 110 and the outer surface of the elongate shaft 12 extending proximally from the end cap 110. When initially disposed around the proximal end region 116 of the end cap 110, the polymer tube 150 may extend across the apertures 122, without material of the tube extending into the apertures 122. A length of heat shrink tubing 160 may be placed over the tube 150 such that the heat shrink tubing 160 surrounds the tube 150 and extends proximally and distally thereof. In some instances, the tube 150 may include a plurality of layers of different polymeric material. For instance, the tube 150 may include a first, inner layer 152 of a first polymer material and a second, outer layer 154 of a second polymer material different from the first polymer material. The inner layer 152 may be compatible with the material of the end cap 110 to facilitate bonding to the end cap 110. For instance, the inner layer 152 may be considered a tie layer, able to form an ionic bond to an oxide layer formed on the end cap 110, formed of stainless steel, for example. In one example, the inner layer 152 may be a maleic anhydride grafted polyethylene (MAGPE), forming a tie layer with the stainless-steel end cap 110. One example of a maleic anhydride grafted polyethylene (MAGPE) is sold by the tradename ReZilok. The outer layer 154 may be a polyamide, such as polyamides sold by the tradename Vestamid®, or other polymeric material.

Heat may then be applied to the tube 150 and the heat shrink tubing 160 to cause the polymer material(s) of the tube 150 to melt and reflow around the proximal end region 116 of the end cap 110 and the outer surface of the elongate shaft 12 extending proximally from the end cap 110. As the polymer material(s) of the tube 150 melt and the heat shrink tubing 160 radially contracts, the polymer material(s) of the tube 150 flow into the apertures 122 and conform to the outer circumferential surface of the end cap 110 and the outer surface of the elongate shaft 12. FIG. 8B illustrates the formed structure subsequent to the reflow process, with the heat shrink tubing 160 removed. As shown, the inner layer 152 may directly contact and confirm to the outer surface of the proximal end region 116 of the end cap 110 to enhance the bond therebetween. For example, the inner layer 152 may form an ionic bond with an oxide layer of the stainless-steel end cap 110, while also bonding to the outer layer 154.

Thereafter, one or more additional layers of polymer material 156 may be disposed over the polymer material(s) of the tube 150 to form a smooth outer surface for the fillet of material 120 transitioning from the outer surface of the elongate shaft 12 to the end cap 110. The additional layer(s) of material 156 may be the same material as the outer layer 154 or a different material compatible with the material of the outer layer 154.

In other instances, the outer circumferential surface of the end cap 110 may be subjected to a surface treatment to enhance the bond between the metal material of the end cap 110 (e.g., stainless-steel) and the polymeric material of the fillet of material 120. For instance, the outer surface of the end cap 110 may be subjected to a silane surface treatment to form a silicon oxide layer that provides a covalent bridge between the stainless-steel of the end cap 110 and the polymer of the fillet of material 120 juxtaposed therewith.

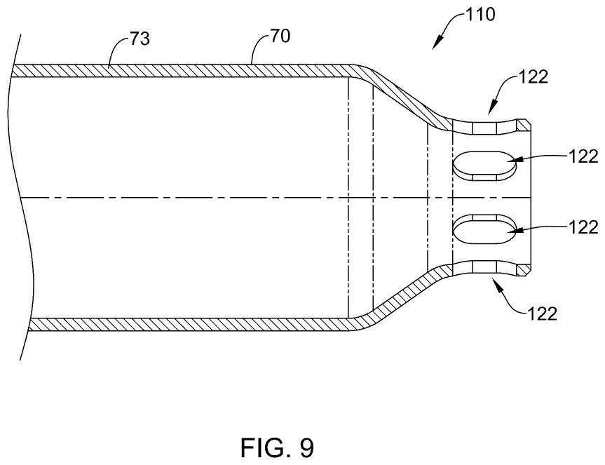

FIG. 9 illustrates an alternative embodiment of the motor housing 70 in which the end cap 110 is formed monolithically with the main body 73 of the motor housing 70. Accordingly, the side wall of the end cap 110 may be continuous with the side wall of the main body 73. The end cap 110 may form a necked down proximal end region of the motor housing 70, having the apertures 122 formed therein. Other configurations are also contemplated.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The scope of the disclosure is, of course, defined in the language in which the appended claims are expressed.

What is claimed is:

1. A percutaneous circulatory support device comprising:
a flexible elongate shaft;
a percutaneous blood pump coupled to a distal end region of the elongate shaft at a junction, the percutaneous blood pump including a blood inlet, a blood outlet, an impeller assembly positioned between the blood inlet and the blood outlet, and a motor housing positioned proximal of the impeller, the motor housing including a motor to rotationally drive an impeller of the impeller assembly to pump blood from the blood inlet to the blood outlet during use;
wherein the motor housing includes an end cap surrounding and secured to a distal end region of the elongate shaft at the junction.

2. The percutaneous circulatory support device of claim 1, wherein the end cap incudes an annular side wall and one or more apertures extending into the annular side wall.

3. The percutaneous circulatory support device of claim 2, wherein the one or more apertures are through holes extending through an entire thickness of the side wall, the through holes having material of the end cap completely surrounding a periphery of the through holes.

4. The percutaneous circulatory support device of claim 2, wherein the one or more apertures are blind holes extending through only a portion of a thickness of the side wall.

5. The percutaneous circulatory support device of claim 2, wherein the one or more apertures are slots extending through an entire thickness of the side wall and opening out to a proximal end of the end cap.

6. The percutaneous circulatory support device of claim 1, wherein the motor housing includes a main body surrounding the motor, and the end cap is a separate component fixed to the main body.

7. The percutaneous circulatory support device of claim 1, wherein the motor housing includes a main body surrounding the motor, and the end cap is formed monolithically with the main body.

8. The percutaneous circulatory support device of claim 1, wherein the end cap includes a distal end region having a first outer diameter and a proximal end region having a second outer diameter, the second outer diameter being less than the first outer diameter.

9. The percutaneous circulatory support device of claim 8, wherein the proximal end region of the end cap includes an annular side wall and one or more apertures extending into the annular side wall.

10. The percutaneous circulatory support device of claim 9, wherein the junction further includes a fillet of material surrounding the proximal end region of the end cap and extending proximally thereof and surrounding an exterior surface of the elongate shaft.

11. The percutaneous circulatory support device of claim 10, wherein the fillet of material extends into the one or more apertures.

12. The percutaneous circulatory support device of claim 11, wherein the end cap includes a shoulder between the proximal end region of the end cap and the distal end region of the end cap, wherein the fillet of material extends distal of the shoulder and surrounds a portion of the distal end region of the end cap.

13. The percutaneous circulatory support device of claim 10, wherein an outer circumferential surface of the proximal end region of the end cap includes one or more grooves, wherein the fillet of material extends into the one or more grooves.

14. The percutaneous circulatory support device of claim 13, wherein the one or more grooves extend circumferentially or helically around the outer circumferential surface.

15. A percutaneous circulatory support device comprising:
a flexible elongate shaft;
a percutaneous blood pump coupled to a distal end region of the elongate shaft at a junction, the percutaneous blood pump configured to pump blood through a housing of the percutaneous blood pump during use;
wherein the junction includes an end cap at a proximal end of the percutaneous blood pump surrounding and secured to a distal end region of the elongate shaft at the junction.

16. The percutaneous circulatory support device of claim 15, wherein the junction further includes a fillet of material surrounding a proximal end region of the end cap and extending proximally thereof and surrounding an exterior surface of the elongate shaft.

17. The percutaneous circulatory support device of claim 16, wherein the proximal end region of the end cap includes an annular side wall and one or more apertures extending into the annular side wall.

18. The percutaneous circulatory support device of claim 17, wherein the fillet of material extends into the one or more apertures and is bonded to the elongate shaft.

19. The percutaneous circulatory support device of claim 18, wherein an outer circumferential surface of the proximal end region of the end cap includes one or more grooves, wherein the fillet of material extends into the one or more grooves.

20. A method of manufacturing a percutaneous circulatory support device, comprising:
inserting a distal end region of an elongate shaft of the percutaneous circulatory support device into a proximal end region of an end cap of a percutaneous blood pump of the percutaneous circulatory support device; and
applying a fillet of material over the proximal end region of the end cap and over a portion of the elongate shaft extending proximally from the end cap to form a junction between the end cap and the elongate shaft.
